(19) United States
(12) Patent Application Publication
(10) Pub. No.: US 2021/0164982 A1
(43) Pub. Date: Jun. 3, 2021

(54) PHARMACEUTICAL USE OF ACTININ-4 INVOLVED IN INDUCTION OF CERVICAL CANCER

(71) Applicant: Korea University Research and Business Foundation, Seoul (KR)

(72) Inventors: Je Sang Ko, Seoul (KR); Hyoung-Tae An, Gyeonggi-do (KR)

(73) Assignee: Korea University Research and Business Foundation, Seoul (KR)

(21) Appl. No.: 17/123,349

(22) Filed: Dec. 16, 2020

Related U.S. Application Data
(62) Division of application No. 15/307,560, filed on Oct. 28, 2016.

(30) Foreign Application Priority Data
Apr. 29, 2014 (KR) 10-2014-0051608

(51) Int. Cl. G01N 33/574; A61K 31/7088; A61P 35/00; C12Q 1/6886; G01N 33/50

(52) U.S. Cl. CPC G01N 33/57411; A61K 31/7088; A61P 35/00; G01N 2333/47; G01N 33/5011; C12Q 2600/136; C12Q 2600/158; C12Q 1/6886

(57) ABSTRACT

The present invention relates to a pharmaceutical use of actinin-4 involved in the induction of cervical cancer, and more specifically, characterizes actinin-4 as a marker protein capable of inducing cervical cancer, thereby providing the pharmaceutical use of actinin-4 that can be used in the diagnosis, prevention or treatment of cervical cancer.

### CROSS-REFERENCE TO RELATED APPLICATIONS

This application is a divisional of U.S. patent application Ser. No. 15/307,560, filed Oct. 28, 2016, which is the U.S. National Phase of International Application No. PCT/KR2015/004160, filed Apr. 27, 2016, which claims priority to Korean Patent Application No. 10-2014-0051608, filed Apr. 29, 2014, the contents of which are incorporated herein by reference in their entirety.

### BACKGROUND

### 1. Field of the Invention

The present invention relates to a pharmaceutical use of actinin-4 as a marker protein for cervical cancer for diagnosis, prevention or treatment of cervical cancer.

### 2. Discussion of Related Art

Epithelial-mesenchymal transition (EMT) refers to a process by which epithelial cells change into mesenchymal cells. That is, the EMT is known as an important process that plays a role in the formation and development of organs in a transition process by which epithelial cells lose their appearance and acquire mesenchymal characteristics. Also, the results from recent studies demonstrated that this process is involved in the progression, invasion, metastasis, etc. of cancer cells.

Actinin-4 known as a cytoskeletal protein is known to be overexpressed in some cancer cells and also involved in migration and growth of cells. However, the role of actinin-4 as a marker protein in cervical cancer remains to not be identified.

Therefore, the present inventors have determined an expression level of actinin-4 in various cancer cells, and identified the roles of actinin-4 in an AKT-Snail signaling pathway when overexpressed in cervical cancer cells; cell proliferation, migration and invasion; and tumorigenesis. Therefore, the present invention has been completed based on these facts.

### SUMMARY OF THE INVENTION

The present invention is directed to providing a pharmaceutical use of actinin-4 for diagnosis, prevention or treatment of cervical cancer by identifying the roles of actinin-4 as a marker protein for cervical cancer.

To solve the above problems, one aspect of the present invention provides a composition for diagnosing cervical cancer, which includes an agent for measuring a level of mRNA of an actinin-4 gene or a protein expressed therefrom.

Another aspect of the present invention provides a pharmaceutical composition for preventing or treating cervical cancer, which includes an actinin-4 inhibitor.

Still another aspect of the present invention provides a use of the actinin-4 inhibitor for preparing the pharmaceutical composition for preventing or treating cervical cancer.

Yet another aspect of the present invention provides a method of treating cervical cancer, which includes administering a pharmaceutically effective amount of the pharmaceutical composition for preventing or treating cervical cancer, which includes an actinin-4 inhibitor, to a subject suffering from cervical cancer.

Yet another aspect of the present invention provides a method of screening a drug for preventing or treating cervical cancer, which includes contacting an actinin-4 gene with a candidate compound outside the human body, and determining whether the candidate compound promotes or inhibits expression of the actinin-4 gene.

Yet another aspect of the present invention provides a method of screening a drug for preventing or treating cervical cancer, which includes contactingan actinin-4 protein with a candidate compound outside the human body, and determining whether the candidate compound promotes or inhibits functions or activities of the actinin-4 protein.

According to the present invention, when actinin-4 which is overexpressed in cervical cancer and involved in cell proliferation, migration and invasion, and tumorigenesis is inhibited, tumorigenesis can be inhibited due to the loss of such effects, and thus the actinin-4 has an effect of being usable for diagnosis, prevention or treatment of cervical cancer when actinin-4 is inhibited.

### BRIEF DESCRIPTION OF THE DRAWINGS

FIGS. 1(A) and 1(B) show: FIG. 1(A) results of comparing expression levels of actinin-4 in various cancer cells (prostate cancer cell lines LNCaP, DU145, and PC3; breast cancer cell lines MCF-7, T47D, and MDA-MB-231; lung cancer cell lines A549, and H460; a colon cancer cell line HCT116; a liver cancer cell line HepG2; and a cervical cancer cell line HeLa, and FIG. 1(B) expression patterns of actinin-4 and E-cadherin in cervical cancer cell lines HeLa, SiHa and ME-180.

FIGS. 2(A), 2(B) and 2(C) show: FIG. 2(A) results of determining an AKT-Snail signaling pathway by overexpression of actinin-4 or inhibition of expression of actinin-4 in a cervical cancer cell line SiHa, and FIG. 2(B) and FIG. 2(C) transcriptional activities of an E-cadherin promoter in the absence or presence of a Snail binding site.

FIG. 3 shows results of preparing cells in which expression of actinin-4 is inhibited and ME-180 is overexpressed in cervical cancer cell lines HeLa and SiHa and comparing expression patterns of E-cadherin and Snail or cell phenotypes in the cells.

FIG. 4 shows migrations of cells when actinin-4 is overexpressed in the cervical cancer cell lines HeLa and SiHa.

FIGS. 5(A) and 5(B) show: FIG. 5(A) migration and FIG. 5(B) invasion of cells in which actinin-4 expression is inhibited in the cervical cancer cell lines HeLa and SiHa.

FIGS. 6(A) and 6(B) show: results of FIG. 6(A) migration/invasion and FIG. (B) cell proliferation of the actinin-4 expression-inhibitory cells in a medium.

FIGS. 7(A), 7(B) and 7(C) show: results of confirming FIG. 7(A) an increase in expression of β-catenin by the overexpression of actinin-4, and FIG. 7(B) and FIG. 7(C) inhibition of degradation and stabilization of β-catenin by actinin-4.

FIGS. 8(A) and 8(B) show: results of confirming FIG. 8(A) inhibition of degradation of proteasomes by β-catenin according to the overexpression of actinin-4, and FIG. 8(B) an increase in transcriptional activities of cyclin D1 as a target protein of β-catenin.

FIGS. 9(A), 9(B) and 9(C) show: results of confirming FIG. 9(A) and FIG. 9(B) decreases in colony formation by the inhibition of expression of actinin-4, and FIG. 9(C) an increase in colony formation by the overexpression of actinin-4.

FIG. 10 shows results of comparing cell growths by the inhibition of expression of actinin-4.

FIG. 11 shows results of confirming inhibition of cell proliferation in the actinin-4 expression-inhibitory cells using PI staining analysis.

FIGS. 12(A) and 12(B) show: FIG. 12(A) formation of tumor at different times in mice into which the actinin-4 expression-inhibitory cells are injected, and FIG. 12(B) images of the mice in which tumors are caused.

FIGS. 13(A), 13(B) and 13(C) show: FIG. 13(A) the types of tumors extracted from the mice into which the actinin-4 expression-inhibitory cells are injected, FIG. 13(B) the sizes and weights of the tumors, and FIG. 13(C) comparison of body weights of the mice after injection of cancer cells.

### DETAILED DESCRIPTION OF EXEMPLARY EMBODIMENTS

Hereinafter, exemplary embodiments of the present invention will be described in detail.

The present invention is directed to providing a composition for diagnosing cervical cancer including an agent for measuring a level of mRNA of an actinin-4 gene or a protein expressed therefrom.

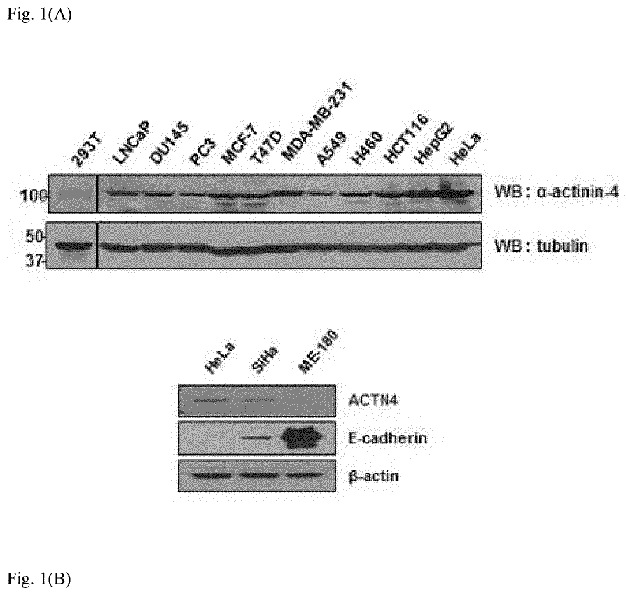

According to one embodiment of the present invention, actinin-4 was overexpressed in a cervical cancer cell line HeLa (FIG. 1A), and thus expression levels of actinin-4 and E-cadherin in cervical cancer cell lines HeLa (HeLa cells are human papillomavirus (HPV)-18-positive), SiHa (SiHa cells are HPV-16-positive), and ME-180 (ME-180 cells are HPV-68-positive) were compared. As a result, the actinin-4 was highly expressed in HeLa cells, but was hardly expressed in ME-180 cells. On the other hand, the E-cadherin was highly expressed in the ME-180 cells in which the actinin-4 was poorly expressed (FIG. 1B).

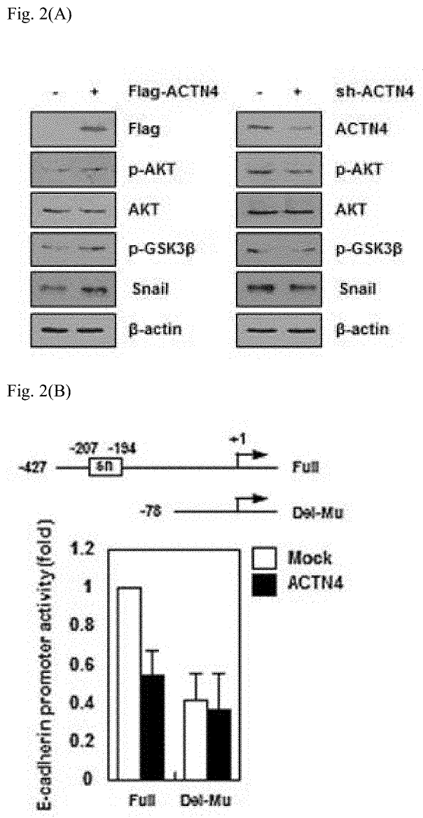

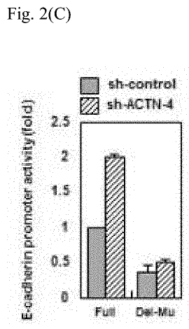

Also, an increase in activation of AKT and an increase in expression of a transcription factor Snail in accordance with an increase in actinin-4 were observed, but the inhibition of expression of actinin-4 resulted in decreased activation of AKT and decreased expression of Snail (FIG. 2A). Since Snail binds to a promoter of E-cadherin to suppress the expression of E-cadherin, transcriptional activities of the E-cadherin promoter were measured. As a result, it was revealed that actinin-4 regulated the expression of E-cadherin by increasing Snail in cervical cancer cells (FIG. 2(B) and FIG. 2(C)).

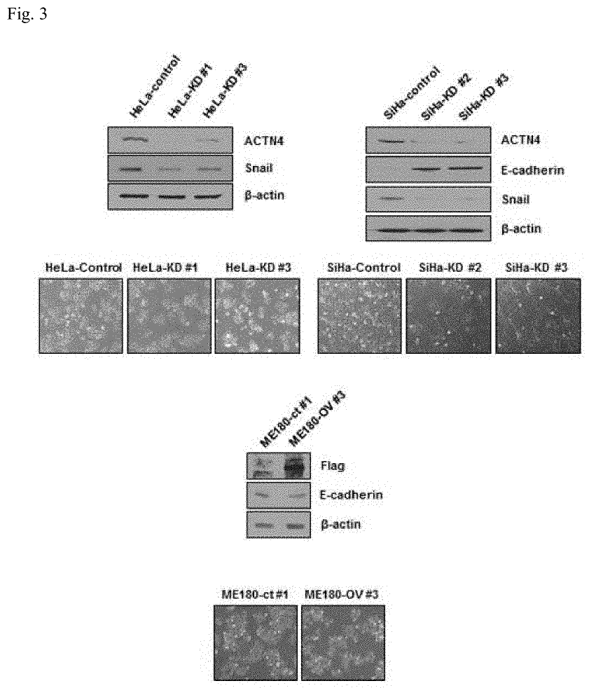

Since actinin-4 was less expressed in the order of HeLa, SiHa, and ME-180 cells, actinin-4 expression-inhibitory cells were prepared from HeLa and SiHa cells, and actinin-4-overexpressing cells were prepared from ME-180 cells in which actinin-4 was hardly expressed, and experiments were performed on both of the prepared cells. As a result, it was revealed that the expression of Snail in the actinin-4 expression-inhibitory cells (e.g., HeLa and SiHa cells) was reduced, and conversely, the expression of E-cadherin increased (FIG. 3). The HeLa cells were cells in which E-cadherin was not expressed, and the expression of E-cadherin did not increase again even when the expression of actinin-4 was inhibited. It seemed to be because actinin-4 was involved in regulating the expression of E-cadherin through Snail expression, but the expression of E-cadherin was also inhibited by other various factors in the cells. Also, the E-cadherin tended to decrease in the ME-180 cells in which actinin-4 was overexpressed. When the phenotype of these cells was checked, it was revealed that the cell phenotype was changed into an MET phenotype in the expression-inhibitory cells whose shape was formed reversely with respect to EMT, that is, a phenotype in which cell-cell binding was altered (the bottom panel of FIG. 3).

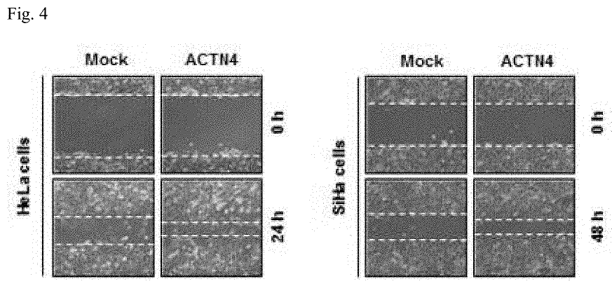

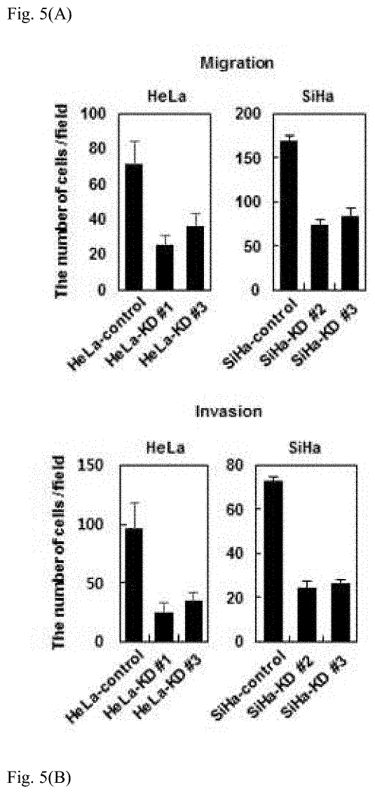

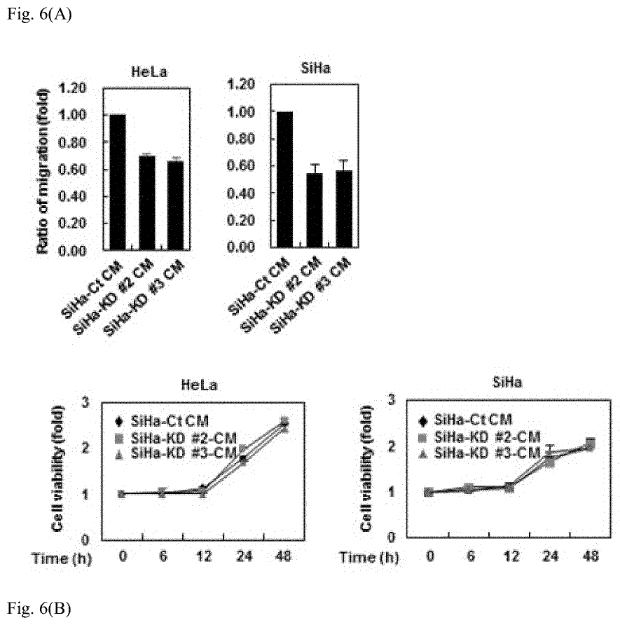

Also, an increase in migration of the cells due to the overexpression of actinin-4 was checked. As a result, it was revealed that the overexpression of actinin-4 resulted in increased migration of the cells in the cervical cancer cell line (FIG. 4). Since the migration and invasion were very important processes for metastasis of cancer cells, transwell migration and MATRIGEL® invasion assays were performed in the actinin-4 expression-inhibitory cells to determine whether the expression of actinin-4 was involved in the migration and invasion of the cancer cells. As a result, the migration of the actinin-4 expression-inhibitory cells decreased, compared to the control cells (FIG. 5A). The results of the MATRIGEL® invasion assay showed that the number of the invaded cells in the actinin-4 expression-inhibitory cells decreased (FIG. 5B). Also, since actinin-4 increased the expression of MMP-9 through Snail, the migration of the cells through the transwell migration in a medium obtained from the actinin-4 expression-inhibitory cells (e.g., SiHa cells) was determined. As a result, it was revealed that the migration of the cells was reduced in the medium obtained from the actinin-4 expression-inhibitory cells, compared to the control medium (FIG. 6A). To determine whether such a decrease in the migration was caused by the proliferation of the cells, an MTT assay was performed using the same medium. As a result, it was revealed that there was no change in the proliferation of the cells in each medium (FIG. 6B). Therefore, it was confirmed that the expression of actinin-4 played an important role as a cancer marker protein in increasing EMT and migration of the cells through a mechanism of increasing AKT-Snail-MMP-9 or inhibiting AKT-Snail-E-cadherin.

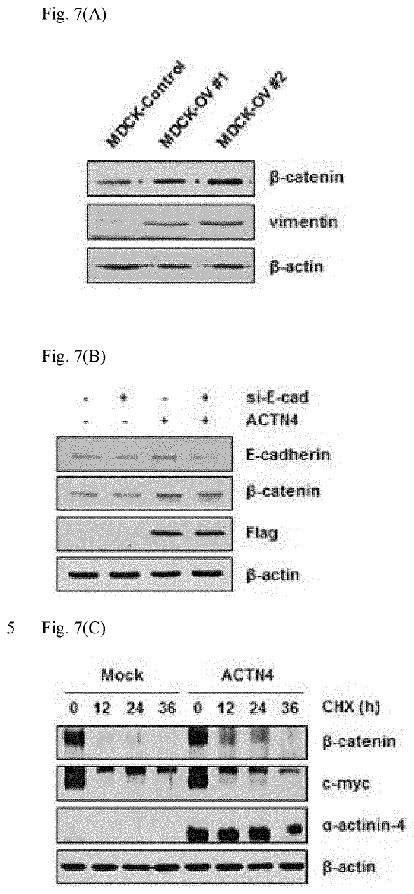

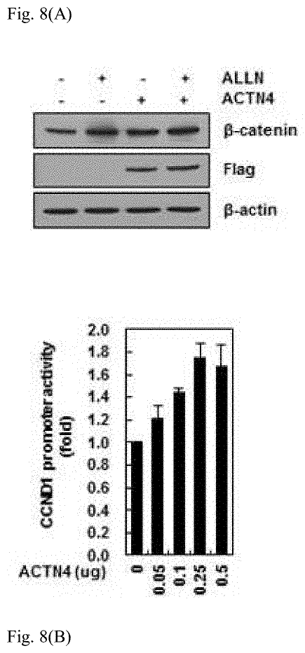

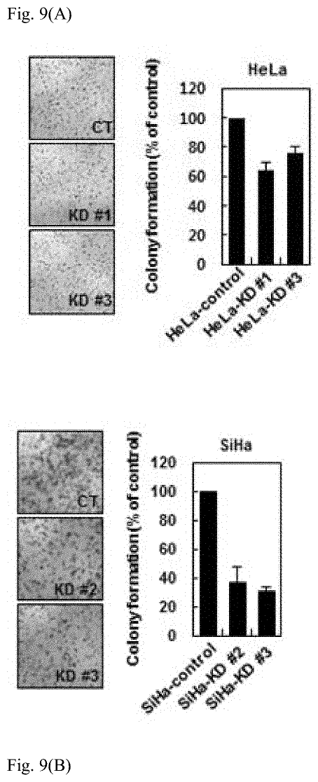

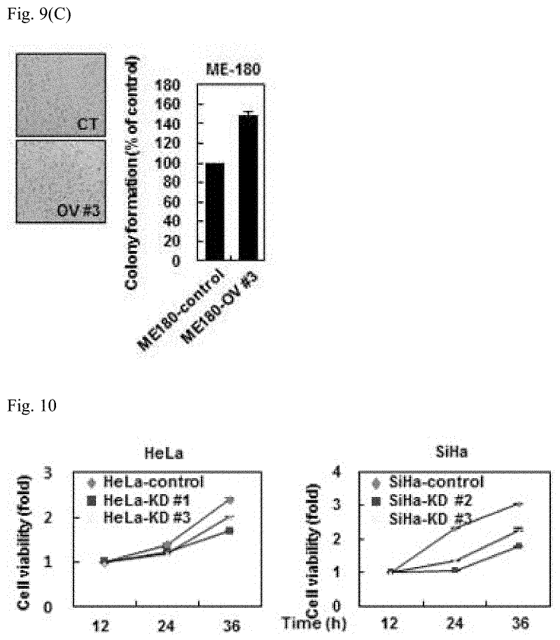

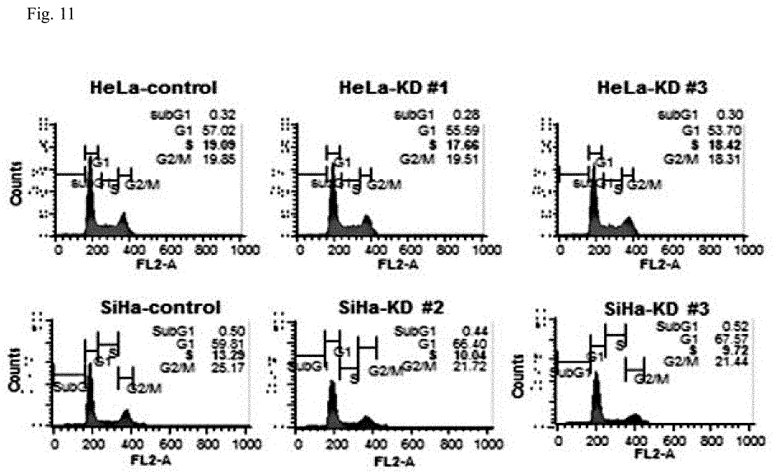

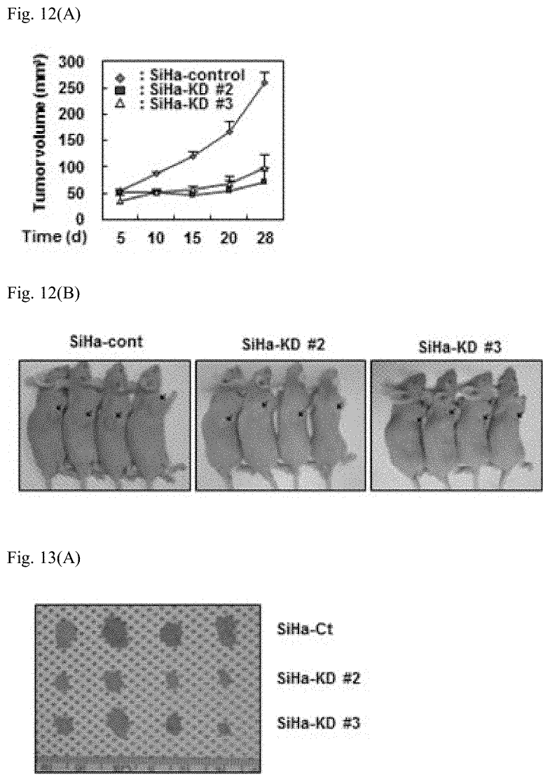

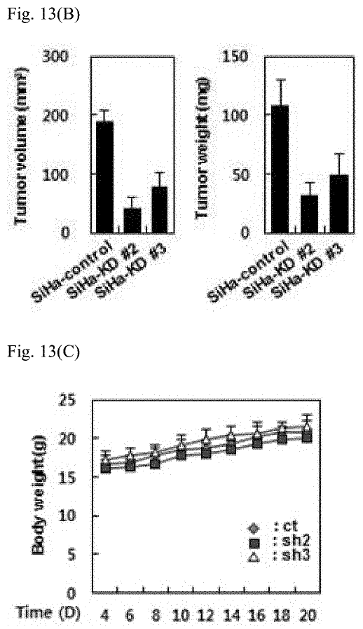

Also, the expression of β-catenin increased in the actinin-4-overexpressing cells (e.g., MDCK cells), and the expression of vimentin as a target protein of β-catenin also increased (FIG. 7A). β-catenin induces the proliferation of cells and is generally bound to E-cadherin which is involved in cell-cell binding. In this case, a decrease in E-cadherin results in decreased β-catenin. However, in the present invention, although E-cadherin disappeared since the expression of E-cadherin was inhibited in the actinin-4-overexpressing cells, the expression of β-catenin increased. Therefore, since an increase in actinin-4 was considered to contribute to stabilization of β-catenin, it was determined, using siRNA, whether the degradation of β-catenin by actinin-4 when the expression of E-cadherin was reduced was inhibited. As a result, a decrease in β-catenin caused by a decrease in E-cadherin was inhibited by the overexpression of actinin-4 (FIG. 7B). Also, the cells were treated with a translation inhibitor (e.g., cycloheximide) to inhibit synthesis of proteins and check whether the degradation of β-catenin was inhibited by actinin-4 at different times. As a result, the degradation of β-catenin was further delayed when actinin-4 was overexpressed, compared to the control (mock), and a decrease in c-myc as a target protein of β-catenin was delayed (FIG. 7C). Since the degradation of β-catenin in the cells was caused by proteasomes, the cells were treated with a proteosome inhibitor (e.g., ALLN) in order to determine whether the degradation of β-catenin caused by degradation of the proteasomes was inhibited to induce the stabilization of β-catenin. As a result, it was revealed that, when actinin-4 was overexpressed, the proteosomal degradation of β-catenin was inhibited and β-catenin was further stabilized with an increased amount of β-catenin (FIG. 8A). Also, the transcriptional activities of cyclin D1 as a target protein of β-catenin were checked. As a result, it was revealed that the activities of cyclin D1 increased depending on a concentration of expressed actinin-4 (FIG. 8B). Since increases in β-catenin and cyclin D1 as the target protein of β-catenin induced the proliferation of cells, the expression of actinin-4 was considered to be involved in the cell proliferation, and thus the cell proliferation was checked using a colony formation assay. As a result, it was revealed that the formation of colonies was inhibited in the actinin-4 expression-inhibitory cell lines HeLa and SiHa (FIGS. 9A and 9B), but the formation of colonies increased in the ME-180 cells in which actinin-4 was overexpressed (FIG. 9C). Therefore, the overexpression of actinin-4 was considered to increase the proliferation of cells, thereby inducing cancer formation. Since the colony formation assay had confirmed that the proliferation of cells was reduced in the actinin-4 expression-inhibitory cells, the growth of the cells was checked using an MTT assay in order to determine whether low expression of actinin-4 was involved in the cell proliferation. As a result, the cell growth was reduced in both of the HeLa and SiHa cells, compared to the control (FIG. 10). From the results of a PI staining assay in which the growth or proliferation of the cells was able to be checked, it was revealed that the percentage of the S-phase cells was reduced in the actinin-4 expression-inhibitory cells HeLa and SiHa, as shown in FIG. 11. From the results, it was seen that actinin-4 functioned to promote the growth of cells transformed into cancer. This was also proven by an animal experiment, that is, the SiHa cells in which the expression of actinin-4 was inhibited were injected into mice to check whether a tumor was formed. As a result, it was revealed that the cancer formation was inhibited in the actinin-4 expression-inhibitory cells, compared to the control cell (FIG. 12A), and that the size of the tumor was also smaller than that of the control (FIG. 12B). Also, the size of the tumor removed from the mice into which the actinin-4 expression-inhibitory cells were injected was smaller than that of the control (FIG. 13A), and the size and weight of the tumor were also significantly reduced, compared to those of the control (FIG. 13B).

Therefore, actinin-4 may be used as a biomarker for diagnosing cervical cancer.

The term “diagnosis” refers to a method of detecting a pathological condition. For the purpose of the present invention, diagnosis means that the onset, development and mitigation of cervical cancer are checked by determining whether a cervical cancer diagnosis marker is expressed.

The term “diagnosis marker” refers to a substance that can distinguish cervical cancer cells from normal cells to diagnose cervical cancer, and includes organic biomolecules such as polypeptides or nucleic acids (for example, mRNA etc.) which tends to increase or decrease in the cervical cancer cells compared to the normal cells, lipids, glycolipids, glycoproteins, saccharides (monosaccharides, disaccharides, oligosaccharides etc.), etc. The cervical cancer diagnosis marker provided in the present invention may be a protein translated from an actinin-4 gene whose expression level increases in the cervical cancer cells, compared to the normal cells.

The composition for diagnosing cervical cancer according to one embodiment of the present invention includes an agent for measuring an expression level of mRNA of an actinin-4 gene or an amount of a protein expressed from the gene. Such an agent includes oligonucleotides having a sequence complementary to the actinin-4 mRNA, for example, a primer or nucleic acid probe specifically binding to the actinin-4 mRNA, or an antibody specific to the actinin-4 protein.

The primer refers to a single-stranded oligonucleotide that can serve as a replication origin for template-directed DNA synthesis at a proper temperature in a proper buffer under proper conditions (that is, four different nucleoside triphosphates and polymerases). A proper length of the primer may vary according to various factors, for example, a temperature and a primer's use. Also, a sequence of the primer does not need to have a sequence completely complementary to some of a sequence of the template, and is enough to have sufficient complementarity in a range in which the primer may be hybridized with a template to fulfill the primer's unique functions. Therefore, in the present invention, the primer does not need to have a sequence completely complementary to a nucleotide sequence of a gene as the template, and is enough to have sufficient complementarity in a range in which the primer may be hybridized with the sequence of the gene to fulfill the primer's functions. Also, the primer according to one embodiment of the present invention should be sufficient to be able to be used in a gene amplification reaction. The amplification reaction refers to a reaction by which nucleic acid molecules are amplified. These gene amplification reactions are widely known in the related art, and may, for example, include a polymerase chain reaction (PCR), a reverse transcriptase-polymerase chain reaction (RT-PCR), a ligase chain reaction (LCR), transcription-mediated amplification (TMA), nucleic acid sequence-based amplification (NASBA), etc.

The nucleic acid probe refers to a natural or modified monomer or a linear oligomer having linkages, and also refers to a naturally existing or artificially synthesized monomer or oligomer that includes a deoxyribonucleotide and a ribonucleotide and can be specifically hybridized with a target nucleotide sequence. The probe according to one embodiment of the present invention may be a single chain, preferably an oligodeoxyribonucleotide. The probe of the present invention may include natural dNMPs (i.e., dAMP, dGMP, dCMP and dTMP), nucleotide analogues or derivatives. Also, the probe of the present invention may also include a ribonucleotide. For example, the probe of the present invention may include backbone-modified nucleotides, for example, peptide nucleic acid (PNA), phosphorothioate DNA, phosphorodithioate DNA, phosphoramidate DNA, amide-linked DNA, MMI-linked DNA, 2′-O-methyl RNA, α-DNA, and methylphosphonate DNA; sugar-modified nucleotides, for example, 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA; and nucleotides having base modifications, for example, a C-5-substituted pyrimidine (whose substituent includes fluoro-, bromo-, chloro-, iodo-, methyl-, ethyl-, vinyl-, formyl-, ethynyl-, propynyl-, alkynyl-, thiazolyl-, imidazolyl-, pyridyl-, etc.), a 7-deazapurine having a C-7 substituent (whose substituent includes fluoro-, bromo-, chloro-, iodo-, methyl-, ethyl-, vinyl-, formyl-, alkynyl-, alkenyl-, thiazolyl-, imidazolyl-, pyridyl-, etc.); inosines; and diaminopurines.

A polyclonal antibody, a monoclonal antibody, a human antibody, and a humanized antibody may be used as the antibody specific to actinin-4.

Examples of the antibody fragment include Fab, Fab′, F(ab′)2 and Fv fragments; a diabody; a linear antibody (Zapata et al., Protein Eng. 8(10): 1057-1062 (1995)); single chain antibody molecules; and a multi-specific antibody derived from the antibody fragment.

When an antibody is degraded by papain, two identical antigen-binding fragments, that is, a “Fab” fragment having a single antigen-binding site, and the other “Fc” fragment are formed. When an antibody is treated with pepsin, an F(ab′)2 fragment that has two antigen-binding sites and can still cross-link with an antigen is formed. Fv is a minimal antibody fragment including intact antigen-recognition and -binding sites. These sites are composed of a dimer including one heavy chain and one light-chain variable region, and are strongly linked via a non-covalent bond.

A method of preparing a polyclonal antibody is known to those skilled in the related art. The polyclonal antibody may be prepared by injecting an immunotherapeutic agent into a mammal one or more times or injecting an immunotherapeutic agent with an immunoadjuvant, when necessary. In general, the immunotherapeutic agent and/or the immunoadjuvant are subcutaneously or intraperitoneally injected into a mammal several times. The immunotherapeutic agent may be a protein according to one embodiment of the present invention, or a fusion protein thereof. It may be effective to inject the immunotherapeutic agent with a protein known to show immunogenicity in the immunized mammal.

The monoclonal antibody according to one embodiment of the present invention may be prepared using a hybridoma method disclosed in Kohler et al., Nature, 256: 495 (1975), or prepared using a recombinant DNA method (for example, see U.S. Pat. No. 4,816,576). Also, the monoclonal antibody may, for example, be isolated from a phage antibody library using a technique disclosed in Clackson et al., Nature, 352: 624-628 (1991) and Marks et al., J. Mol. Biol., 222: 581-597(1991).

Specifically, when the monoclonal antibody according to one embodiment of the present invention exhibits desired activities, the monoclonal antibody includes “chimeric” antibodies in which a part of a heavy chain and/or a light chain has a corresponding sequence identical to antibodies derived from certain species or antibodies belonging to a certain class or subclass of antibodies, or shows homology with the antibodies and in which the other part of the chain(s) has a sequence identical to antibodies derived from the other species, antibodies belonging to other class or subclass antibodies or fragments thereof, or shows homology with the antibodies or fragments thereof (Morrison et al., Proc. Natl. Acad. Sci. USA, 81: 6851-6855 (1984)).

A “humanized” form of a non-human (for example, rodent) antibody is a chimeric immunoglobulin, an immunoglobulin chain or fragments thereof (for example, Fv, Fab, Fab′, F(ab′)2, or another antigen-binding sequence of the antibody), all of which have a minimal sequence derived from a non-human immunoglobulin. In most cases, the humanized antibody includes a human immunoglobulin (a recipient antibody) in which a complementarity determining region (CDR) residue of a recipient is substituted with a CDR residue derived from species (donor antibodies) excluding human beings, such as a rat, a mouse or a rabbit, which have desired specificity, affinity and potency. In some cases, Fv framework residues of the human immunoglobulin are substituted with the corresponding non-human residues. Also, the humanized antibody may include a recipient antibody, or residues not found in a CDR or framework sequence to be introduced. In general, the humanized antibody includes substantially all of one or more, generally two or more variable domains. Here, all or substantially all of the CDR regions correspond to regions of the non-human immunoglobulin, and all or substantially all of the FR regions correspond to regions of a human immunoglobulin sequence. Also, the humanized antibody includes at least a part of an immunoglobulin constant region (Fc), generally a part of a human immunoglobulin region (Presta, Curr. Op. Struct. Biol. 2: 593-596 (1992)).

The composition for diagnosing cervical cancer according to the present invention may be included in the form of a kit.

The kit may be include a primer, a probe or an antibody, all of which can be used to measure an expression level of an actinin-4 gene or an amount of a protein, and definitions thereof are as described above.

When the kit is applied to a PCR amplification process, the kit may optionally include reagents required for PCR amplification, for example, a buffer, a DNA polymerase (for example, a heat-stable DNA polymerase obtained from Thermus aquaticus (Taq), Thermus thermophilus (Tth), Thermus filiformis, Thermis flavus, Thermococcus literalis, or Pyrococcus furiosus (Pfu)), a DNA polymerase cofactor, and dNTPs. When the kit is applied to an immunoassay, the kit of the present invention may optionally include a secondary antibody and a labeled substrate. Further, the kit according to the present invention may be manufactured as a number of separate packages or compartments including the above-described reagent ingredients.

Also, the composition for diagnosing cervical cancer according to one embodiment of the present invention may be included in the form of a microarray.

In the microarray of the present invention, the primer, probe or antibody which can be used to measure an expression level of the actinin-4 protein or the gene coding for the protein may be used as a hybridizable array element, and is fixed on a substrate. A preferred substrate may include a proper rigid or semi-rigid support, for example, a membrane, a filter, a chip, a slide, a wafer, fibers, magnetic or non-magnetic beads, a gel, a tubing, a plate, a polymer, microparticles, and a capillary tube. The hybridizable array element may be arranged and fixed on the substrate, such fixation may be performed using a chemical bonding method or a covalent bonding method using UV rays. For example, the hybridizable array element may be bound to a surface of a glass modified to contain an epoxy compound or an aldehyde group, and may also be bound to a polylysine-coated surface using UV rays. Also, the hybridizable array element may be bound to a substrate via a linker (e.g., an ethylene glycol oligomer and a diamine).

Meanwhile, when a sample applied to the microarray of the present invention is a nucleic acid, the sample may be labeled, and hybridized with an array element on the microarray. There may be various hybridization conditions, and the detection and analysis of a hybridization level may be widely performed according to a labeled substance.

Also, the present invention is directed to providing a method of diagnosing cervical cancer using the method of measuring an expression level of the actinin-4 gene or a level of the protein expressed therefrom. More specifically, the method may include (a) measuring an expression level of an actinin-4 gene from a biological sample of a suspected cervical cancer patient or an amount of a protein expressed therefrom; and (b) measuring an expression level of the gene from a normal control sample or an amount of the protein expressed therefrom to compare the measured results to the measured results of (a).

As such, a method of measuring an expression level of the gene and an amount of the protein includes a known process of separating mRNA or a protein from a biological sample, and thus may be performed using a known technique.

The biological sample refers to a sample collected from a biological body in which the expression level of the gene and the amount of the protein are different from the normal control, depending on the onset or progression of cervical cancer. For example, the sample may include tissues, cells, blood, serum, plasma, saliva, and urine, but the present invention is not limited thereto.

The measurement of the expression level of the gene preferably includes measuring a level of mRNA. Here, a method of measuring a level of mRNA includes a reverse transcriptase-polymerase chain reaction (RT-PCR), a real-time reverse transcriptase-polymerase chain reaction, an RNase protection assay, Northern blotting, and a DNA chip assay, but the present invention is not limited thereto.

The measurement of the amount of the protein may be performed using an antibody. In this case, the actinin-4 protein in the biological sample and an antibody specific to the protein form a conjugate, that is, an antigen-antibody complex, and an amount of the formed antigen-antibody complex may be quantitatively measured based on the signal intensity of a detection label. Such a detection label may be selected from the group consisting of an enzyme, a fluorescent material, a ligand, a luminescent material, microparticles, redox molecules, and a radioisotope, but the present invention is not limited thereto. An analysis method of measuring an amount of the protein includes Western blotting, ELISA, a radioimmunoassay, radioimmunodiffusion, Ouchterlony immunodiffusion, rocket immunoelectrophoresis, immunohistological staining, an immunoprecipitation assay, a complement fixation assay, FACS®, a protein chip assay, etc., but the present invention is not limited thereto.

Therefore, in the present invention, the expression level of mRNA or the amount of the protein of the control, and the expression level of mRNA or the amount of the protein from a cervical cancer patient or a suspected cervical cancer patient may be determined using the above-described detection methods. In this case, the onset and progression stage of cervical cancer may be diagnosed by comparing the expression level to that of the control.

Also, the method of diagnosing cervical cancer according to one embodiment of the present invention may be used to judge that cervical cancer is caused when the expression level of the actinin-4 gene according to the present invention and the amount of the protein expressed therefrom increase, compared to the normal control sample.

In addition, the present invention is directed to providing a pharmaceutical composition for preventing or treating cervical cancer, which includes an actinin-4 inhibitor.

Additionally, the present invention is directed to providing a use of the actinin-4 inhibitor for preparing the pharmaceutical composition for preventing or treating cervical cancer.

According to the present invention, actinin-4 is overexpressed in the cervical cancer cell, as described above, and the expression of actinin-4 induces cancer formation by increasing EMT and migration of cells and inhibiting the degradation of β-catenin involved in the proliferation of the cells to delay a decrease in c-myc as a target protein of β-catenin through a mechanism of increasing AKT-Snail-MMP-9 or inhibiting AKT-Snail-E-cadherin, and increasing the transcriptional activities of cyclin D1 to increase the cell proliferation. However, when the expression of actinin-4 is inhibited, the cells have an MET phenotype, that is, a phenotype in which cell-cell binding is altered, the migration and invasion of the cells decreases, the cell proliferation is reduced, the tumor formation is inhibited in an animal model, and the size of the formed tumor is also smaller compared to the control. Therefore, the actinin-4 inhibitor may be used to treat cervical cancer.

Accordingly, the composition for preventing or treating cervical cancer according to one embodiment of the present invention may include an agent capable of reducing the expression of mRNA of the actinin-4 gene or the expression of the protein expressed therefrom, or reducing the functions or activities of the actinin-4 protein.

The actinin-4 protein inhibitor may be a peptide or compound that binds to the actinin-4 protein to regulate signals from a neuronal differentiation pathway. Such an inhibitor may be selected using a screening method as will be described below, such as protein structure analysis, and may be designed using methods known in the related art.

Also, a polyclonal antibody, a monoclonal antibody, a human antibody and a humanized antibody against the actinin-4 protein may be used as the protein inhibitor, and definitions of the antibodies are as described above.

Cervical cancer may be prevented or treated by inhibiting the functions of actinin-4 in the cells using the antibody.

The inhibitor used to inhibit the functions and activities of the actinin-4 protein of the present invention may be transferred using a liposome, a virus, a gene gun, a polymer, ultrasonication, electric shock, etc., but the present invention is not particularly limited thereto.

The actinin-4 gene may be DNA coding for the actinin-4 gene, or mRNA transcribed from the DNA. Therefore, the inhibitor of the gene may be an inhibitor that binds to the gene itself to inhibit transcription of the gene or binds to mRNA transcribed from the gene to inhibit translation of mRNA.

Therefore, the inhibitor of the actinin-4 gene includes all types of inhibitors that inhibit the expression of the actinin-4 gene. For example, such an inhibitor may be a peptide, a nucleic acid or a compound that binds to the gene. Such an inhibitor may be selected using a screening method as will be described below, such as cell-based screening, and may be designed using methods known in the related art.

According to one embodiment, the inhibitor may be an anti-sense-oligonucleotide against the actinin-4 gene, siRNA, shRNA, miRNA, or a vector including the same. The anti-sense-oligonucleotide, siRNA, shRNA, miRNA or the vector including the same may be constructed using methods known in the related art.

As used herein the term “siRNA” refers to a double-stranded RNA that induces RNA interference through the cleavage of mRNA of a target gene, and is composed of an RNA strand having the same sense sequence as the mRNA of the target gene and an RNA strand having an anti-sense sequence complementary to the sense sequence.

The siRNA may include types of in vitro synthesized siRNA which are expressed when the siRNA itself or a base sequence coding for the siRNA is inserted into an expression vector.

As used herein the term “vector” refers to a gene construct that contains exogenous DNA inserted into the genome to code for a polypeptide.

The vector related to the present invention may be a vector in which a nucleic acid sequence inhibiting the gene is inserted into the genome, and examples of the vector may include a DNA vector, a plasmid vector, a cosmid vector, a bacteriophage vector, a yeast vector, or a viral vector.

Also, the anti-sense sequence has a sequence complementary to all or some of the mRNA sequence transcribed from the actinin-4 gene or fragments thereof, and may bind to the mRNA to inhibit the expression of the actinin-4 gene or fragments thereof.

In addition, since the short hairpin RNAi (shRNAi) targets a shRNAi consensus sequence region of a human being or a mouse, the shRNAi constructed by conventional methods may be used.

Further, the pharmaceutical composition according to one embodiment of the present invention may further include a pharmaceutically acceptable carrier.

The pharmaceutically acceptable carrier includes carriers and vehicles generally used in the field of drugs. Specifically, the pharmaceutically available carrier includes an ion exchange resin, alumina, aluminum stearate, lecithin, a serum protein (for example, human serum albumin), a buffer (for example, various phosphates, glycine, sorbic acid, potassium sorbate, and a partial glyceride mixture of saturated vegetable fatty acid), water, a salt or electrolyte (for example, protamine sulfate, disodium hydrogen phosphate, potassium hydrogen phosphate, sodium chloride, and zinc salt), colloidal silica, magnesium trisilicate, polyvinylpyrrolidone, a cellulose-based substrate, polyethylene glycol, sodium carboxymethylcellulose, a polyacrylate, wax, polyethylene glycol, wool fat, etc., but the present invention is not limited thereto.

Also, the composition according to one embodiment of the present invention may further include a lubricating agent, a wetting agent, an emulsifying agent, a suspending agent, or a preservative in addition to the above-described ingredients.

According to one embodiment, the composition of the present invention may be prepared as a water-soluble solution for parenteral administration. Preferably, a buffer solution such as Hank's solution, Ringer's solution, or a buffer solution such as physically buffered saline may be used. A substrate capable of enhancing the viscosity of a suspension, such as sodium carboxymethylcellulose, sorbitol, or dextran, may be added to a water-soluble injectable suspension.

The composition according to one embodiment of the present invention may be systemically or topically administered, and may be prepared into proper formulations using known techniques for such administration. For example, the composition may be mixed with an inert diluent or an edible carrier upon oral administration, sealed in a hard or soft gelatin capsule, or pressed into tablets to be administered. For oral administration, an active compound may be mixed with an excipient, and may then be used in the form of an intake-type tablet, a buccal tablet, a troche, a capsule, an elixir, a suspension, syrup, wafer, etc.

Various formulations for injection and parenteral administration may be prepared using techniques known in the related art or techniques generally used in the related art. After actinin-4 is stored in a freeze-dried state since the actinin-4 is easily dissolved in saline or a buffer, an effective amount of the actinin-4 may be prepared into a solution immediately before being added to saline or a buffer, and then administered in a form suitable for intravenous injection, subcutaneous injection, intramuscular injection, intraperitoneal injection, percutaneous administration, etc.

An effective amount of the active ingredient of the pharmaceutical composition according to one embodiment of the present invention refers to an amount required to achieve an effect of preventing, suppressing and alleviating diseases.

Therefore, the effective amount of the active ingredient may be adjusted according to various factors including the kind of a disease, the severity of a disease, the kinds and contents of the active ingredient and other ingredients included in the composition, the kind of a formulation, the age, body weight, general physical condition, sex and diet of a patient, the time and route of administration, the secretion rate of the composition, the duration of treatment, and drugs used together. The inhibitor according to one embodiment of the present invention may, for example, be administered to an adult once or several times per day. In this case, a compound may be administered at a dose of 0.1 ng/kg to 10 g/kg, a polypeptide, a protein or antibody may be administered at a dose of 0.1 ng/kg to 10 g/kg, and an anti-sense-oligonucleotide, siRNA, shRNAi, or miRNA may be administered at a dose of 0.01 ng/kg to 10 g/kg, but the present invention is not limited thereto.

Also, the present invention is directed to providing a method of treating cervical cancer, which includes administering a pharmaceutically effective amount of the pharmaceutical composition for preventing or treating cervical cancer, which includes an actinin-4 inhibitor, to a subject suffering from cervical cancer.

The pharmaceutical composition and the administration method used in the method of treating cervical cancer are described above, and thus descriptions of the common contents between both of the methods are omitted to avoid excessive complexity in this specification.

Meanwhile, the subject to which the pharmaceutical composition for preventing or treating cervical cancer may be administered includes all kinds of animals. For example, the subject may be an animal except a human being, including a dog, a cat, a rat, and the like.

In addition, the present invention is directed to providing a method of screening a drug for preventing or treating cervical cancer, which includes contacting an actinin-4 gene with a candidate compound outside the human body, and determining whether the candidate compound promotes or inhibits expression of the actinin-4 gene.

Further, the present invention is directed to providing a method of screening a drug for preventing or treating cervical cancer, which includes contacting an actinin-4 protein with a candidate compound outside the human body, and determining whether the candidate compound promotes or inhibits functions or activities of the actinin-4 protein.

According to the screening method of the present invention, first of all, a candidate compound to be analyzed may be brought into contact with cervical cancer cells including the gene or the protein.

The candidate compound may include individual nucleic acids, proteins, peptides, other extracts or natural products, and compounds, all of which are assumed to have a probability as a substance that promotes and inhibits transcription of a base sequence of the actinin-4 gene into mRNA and translation of the mRNA into a protein or a drug that promotes and inhibits the functions and activities of the actinin-4 protein, or randomly selected according to a conventional selection method.

Next, an expression level of the gene and an amount or activities of the protein in cells treated with the candidate compound may be measured. From the measured results, when the expression level of the gene and the amount or activities of the protein are determined to have been increased or decreased, the candidate compound may be contemplated as a substance capable of treating or preventing cervical cancer.

As such, a method of measuring an expression level of the gene and an amount or activities of the protein may be performed using various methods known in the related art. For example, the method may be performed using a reverse transcriptase-polymerase chain reaction, a real-time polymerase chain reaction, Western blotting, Northern blotting, an enzyme-linked immunosorbent assay (ELISA), a radioimmunoassay (RIA), radioimmunodiffusion, and an immunoprecipitation assay, but the present invention is not limited thereto.

The candidate compound obtained by the screening method of the present invention, which has an activity to inhibit the expression of the gene or suppress the functions of the protein, may be used as a therapeutic candidate compound for treating cervical cancer.

Such a therapeutic candidate compound for treating cervical cancer serves as a leading compound during subsequent development of cervical cancer therapeutics, and thus novel cervical cancer therapeutics may be developed by modifying and optimizing a structure of the leading compound in order to show an effect of promoting or inhibiting the functions of the actinin-4 gene or the protein expressed therefrom.

The contents related to the genetic engineering technology in the present invention will become more apparent from the contents disclosed in Sambrook, et al. Molecular Cloning, A Laboratory Manual, Cold Spring Harbor Laboratory Press, Cold Spring Harbor, N. Y. (2001) and Frederick M. Ausubel et al., Current Protocols in Molecular Biology volumes 1, 2, 3, John Wiley & Sons, Inc. (1994).

These and other advantages and features of the present invention and method of achieving them will be apparent from the description of the following examples, with reference to the accompanying drawings. However, the present invention is not limited to the following examples and can be exemplified in various forms. That is, the examples of the present invention serve to complete the disclosure of the present invention, and are provided to inform a person who has an ordinary knowledge and skill in the art to which this invention belongs of the full scope of the invention. This invention should be defined based on the scope of the appended claims.

Hereinafter, the commercially available reagents, media and the like used in the experiments are as follows:

A Dulbecco's modified Eagle's medium (DMEM), fetal bovine serum (FBS), penicillin, streptomycin, and a LIPOFECTAMINE® 2000 reagent were purchased from Invitrogen (Carlsbad, Calif.).

Anti-ACTN4, anti-vimentin, anti-N-cadherin, anti-β-actin and anti-AKT1 antibodies were purchased from Santa Cruz Biotechnology, Inc. (Santa Cruz, Calif.), anti-p-GSK3β, anti-Snail and anti-p-AKT (S473) antibodies were purchased from Cell Signaling Technology, and anti-E-cadherin and anti-β-catenin antibodies were purchased from BD Biosciences. Monoclonal anti-Flag-M2 antibodies were purchased from Aligent Technology.

A sh-ACTN4 plasmid was purchased from Open Biosystems.

Trypan blue and 3-(4,5-dimethylthiazol-2-yl)-2,5-diphenyltetrazolium bromide (MTT) were purchased from Sigma (St. Louis, Mo.).

### <Example 1> Comparison of Expression of Actinin-4 in Various Cancer Cells and Expression of Actinin-4 in Cervical Cancer Cells

To compare expression levels of actinin-4 in various cancer cells, expression levels of the protein were compared using Western blotting.

For this purpose, the expressions of actinin-4 in various cancer cell (prostate cancer cell lines LNCaP, DU145, and PC3; breast cancer cell lines MCF-7, T47D, and MDA-MB-231; lung cancer cell lines A549, and H460; a colon cancer cell line HCT116; a liver cancer cell line HepG2, and a cervical cancer cell line HeLa) including a human embryonic kidney cell line (HEK-293T) as a conventional cell line were compared. The amount of the protein in each cell line was 30 μg, a 10% SDS-PAGE gel was used, and an antibody specific to the actinin-4 protein (Santa Cruz) was used at a ratio of 1:3,000. For independent protein detection, the protein was confirmed in a dark room using WEST PICO® ECL (Thermo scientific, Rockford, Ill.). A tubulin antibody (Santa Cruz) was treated at a ratio of 1:3,000 as the control used to determine whether the protein was expressed at the same levels in each cell line.

As shown in FIG. 1A, an excessive amount of actinin-4 was expressed in the cervical cancer cell line HeLa, compared to the other cancer cell lines.

Based on the results, the expression levels of actinin-4 in the cervical cancer cell lines HeLa, SiHa and ME-180 were compared, and the expression of E-cadherin according to the expression levels was confirmed.

For this purpose, 30 μg of the protein obtained from cervical cancer cells included in three cervical cancer cell lines (HeLa, SiHa, and ME-180) was Western-blotted to compare the expression levels of the actinin-4 and E-cadherin proteins (BD Biosciences). An E-cadherin antibody was used at a ratio of 1:5,000.

As shown in FIG. 1B, actinin-4 was highly expressed in the HeLa cells, and hardly expressed in the ME-180 cells. On the other hand, E-cadherin was highly expressed in the ME-180 cells in which actinin-4 was poorly expressed.

From these results, it was shown that the expression of E-cadherin was regulated due to the overexpression of actinin-4.

### <Example 2> Examination of AKT-Snail Signaling Pathway and Transcriptional Activities of E-Cadherin by Expression of Actinin-4 in Cervical Cancer Cells

In a previous research report, an AKT-Snail signaling pathway by the expression of actinin-4 in MDCK cells was identified. Therefore, it was checked whether the same signaling pathway occurred in the cervical cancer cells.

For this purpose, SiHa cells were transfected with flag-actinin-4 DNA (1 μg) or sh-actinin-4 (1.5 μg) using LIPOFECTAMINE® 2000 (Invitrogen). The DNA and LIPOFECTAMINE® 2000 were used at a ratio of 1:2, and a method was performed according to the manufacturer's manual. The sh-actinin-4 was purchased from Open Biosystems and used. After proteins were extracted from the corresponding cells using an RIPA lysis buffer, 30 μg of the proteins were Western-blotted with each of FLAG (Aligent Technology), p-AKT (Cell Signaling Technology), AKT (Santa Cruz), p-GSK3β and Snail (Cell Signaling Technology) and β-actin (Santa Cruz) antibodies to determine expression levels of the proteins. Here, each of the antibodies was used at a ratio of 1:3,000.

Also, HEK-293T cells were seeded in a 12-well plate at 2×10⁵ cells/well, and transfected with 0.5 μg of an E-cadherin promoter construct (having a full-length size of −427 to +53; Del-mutant: −78 to +53) and Flag-ACTN4 (0.5 μg) or sh-ACTN4 (0.5 μg) at the same time. The deletion mutant was constructed by cloning a −78^{th} to +53^{rd} region other than a −207^{th} to −194^{th} region including a Snail binding site into a full-length promoter. Sn represents a binding site (−207 to −194) of Snail. The corresponding cells were washed with ice-cold PBS, lysed in a reporter lysis buffer (Promega, Madison, Wis.) at 80 μl per well, and then centrifuged at 4° C. and 10,000 g for 10 minutes to collect a supernatant. Then, the supernatant was measured for luciferase activity. Luminometer 20/20ⁿ (Turner Biosystems, Sunnyvale, Calif.) was used as a measuring system. For normalization, the cells were co-transfected together with pSV40-β-galactosidase. The activities of β-galactosidase in the collected supernatant were measured to correct the luciferase activities, which were then plotted in a graph. Here, a β-galactosidase analysis system (Promega, Madison, Wis.) was used, and a DU530 spectrophotometer (Beckman Instruments, Palo Alto, Calif.) was used for analysis.

As shown in FIG. 2A, it was revealed that an increase in activation of AKT and an increase in expression of Snail in accordance with an increase in actinin-4 were observed, but the inhibition of expression of actinin-4 also caused a decrease in the activation of AKT and a decrease in the expression of Snail.

The transcription factor Snail serves to bind to a promoter of E-cadherin to inhibit the expression of E-cadherin. Therefore, the transcriptional activities of two E-cadherin promoters with or without a binding site of Snail were measured. As a result, it was revealed that the transcriptional activities in the promoter including the Snail binding site were reduced, and the transcriptional activities were not varied regardless of the presence or absence of actinin-4 when the Snail binding site was removed (FIG. 2B). Also, when the expression of actinin-4 was inhibited, the transcriptional activities of E-cadherin increased (FIG. 2C).

From these results, it was shown that actinin-4 regulated the expression of E-cadherin by increasing Snail in the cervical cancer cells.

### <Example 3> Examination of Expression Patterns of Snail and E-Cadherin by Construction of Actinin-4 Expression-Inhibitory Cells and Actinin-4-Overexpressing Cells from Cervical Cancer Cell Lines HeLa, SiHa and ME-180

In Example 1, actinin-4 was less expressed in the order of HeLa, SiHa, and ME-180 cells. Therefore, actinin-4 expression-inhibitory cells were prepared from HeLa and SiHa cells, and actinin-4-overexpressing cells were prepared from ME-180 cells in which the actinin-4 was hardly expressed.

To construct actinin-4 expression inhibition stabilizing cells, HeLa and SiHa cells were transfected with an shACTN4 plasmid (1.5 μg), and then selected in the presence of puromycin (2 μg/mL) for 2 weeks. Proteins were extracted from the corresponding cells, and the expression levels of the actinin-4 and Snail proteins were then determined using Western blotting. Also, to construct actinin-4 overexpression stabilizing cells in the ME-180 cells, the ME-180 cells were transfected with a Flag-ACTN4 plasmid, and selected by treatment with G-418 (400 μg/mL; Sigma) for 2 weeks. Images of the corresponding cells were taken under a microscope to determine the phenotype (a cell shape) of the corresponding cells (Zeiss Axiovert100; Magnification: 10×).

As shown in FIG. 3, it was revealed that the expression of Snail was reduced in the actinin-4 expression-inhibitory cells such as HeLa and SiHa cells, and conversely, the expression of E-cadherin increased. The HeLa cells were cells in which E-cadherin was not expressed, and the expression of E-cadherin did not increase again even when the expression of actinin-4 was inhibited. It seemed to be because actinin-4 was involved in regulating the expression of E-cadherin through Snail expression, but the expression of E-cadherin was also inhibited by other various factors in the cells.

Next, the E-cadherin tended to decrease in the ME-180 cells in which actinin-4 was overexpressed. When the phenotype of these cells was checked, it was revealed that the cell phenotype was changed into an MET phenotype in the expression-inhibitory cells whose shape was formed reversely with respect to EMT, that is, a phenotype in which cell-cell binding was altered (the bottom panel of FIG. 3).

### <Example 4> Examination of Migration of Cells Due to Overexpression of Actinin-4 in Cervical Cancer Cells

To determine the migration of cells due to the overexpression of actinin-4 in the cervical cancer cells, a wound healing assay was performed.

To perform the wound healing assay, HeLa and SiHa cells were seeded in a 6-well plate at 5×10⁵ cells/well, and then transfected with actinin-4 (1 μg). Thereafter, the cells in each well were scratched in a straight line using a 200p tip, and migration degrees of the cells on a surface of each well toward a location from which the cells were removed were compared (HeLa for 24 hours, and SiHa for 48 hours).

As shown in FIG. 4, it was revealed that the overexpression of actinin-4 increased the migration of the cells.

The migration and the invasion are very important processes for metastasis of cancer cells. Therefore, transwell migration and MATRIGEL® invasion assays were performed in the actinin-4 expression-inhibitory cells to determine whether the expression of actinin-4 was involved in the migration and invasion of the cancer cells.

To measure the migration of the cells, the transwell migration was performed using actinin-4 expression inhibition stabilizing cells (HeLa-KD, and SiHa-KD). A transwell insert (BD Biosciences) with holes having a pore size of 8 μm was coated with collagen I (20 μg) for an hour. Thereafter, HeLa cells (the control and KD cells: 3×10⁵) and SiHa cells (the control and KD cells: 5×10⁵) were seeded in the insert, and the number of the cells migrating to the other side of the pores after 36 hours was counted, and then plotted in a graph.

As shown in FIG. 5A, it was revealed that the migration of the actinin-4 expression-inhibitory cells was reduced, compared to the control cells.

Since cells in one cancer have an ability to penetrate into blood vessels or tissues when the cells in the cancer tissue metastasize into other biological organs, the MATRIGEL® invasion assay is an experiment used to check such an ability of the cells in the cancer tissue. The number of cells migrating to the other side of a membrane through MATRIGEL® was counted.

For this purpose, an insert plate (BD Biosciences) with holes having a pore size of 8 μm was coated with MATRIGEL® (2 mg/mL; BD Biosciences) for an hour. Thereafter, HeLa cells (the control and KD cells: 5×10⁵) and SiHa cells (the control and KD cells: 5×10⁵) were seeded in the insert plate, and the number of the cells migrating to the other side of the pores after 36 hours was counted, and then plotted in a graph.

As shown in FIG. 5B, it was revealed that the number of the invading cells in the actinin-4 expression-inhibitory cells was reduced.

Also, since actinin-4 increased the expression of MMP-9 through Snail, the migration of the cells was also thought to be inhibited in a medium obtained from the actinin-4 expression-inhibitory cells. Therefore, the migration of the cells through the transwell migration in the medium obtained from the actinin-4 expression-inhibitory cells (e.g., SiHa cells) was checked.

For this purpose, the HeLa and SiHa cells were treated with a conditioned medium (CM) obtained from the actinin-4 expression-inhibitory cells, and the migration of the cells was tested using a transwell migration method. After the actinin-4 expression-inhibitory cells SiHa were seeded in a 6-well plate at 5×10⁵ cells/well, the cells were treated with serum-free DMEM for 48 hours, and the CM was then collected and centrifuged at 500 g for 5 minutes to remove the residual cells and recover the medium only. A cell migration test method was performed in the same manner as in the above-described migration test.

Also, to determine whether the corresponding medium (CM) was involved in the growth of the cells, the HeLa and SiHa cells were seeded in a 96-well plate at 1×10⁴ cells/well, and then treated with the CM (the control CM or ACTN4-KD CM) obtained from the SiHa-actinin-4 expression-inhibitory cells. Then, the number of the cells at different times (0 to 48 hours) was counted, and plotted in a graph. Here, each experiment was performed in triplicate. After each well was treated with 100 μl of an MTT solution (5 mg/mL, Sigma) for 6 hours, 100 mL of dimethyl sulfoxide (DMSO) was added to stop the reaction. The measurement was performed at a wavelength of 590 nm using an ELISA reader (Bio-Rad Laboratories, Inc.).

As shown in FIG. 6A, it was revealed that the migration of the cells was reduced in the medium obtained from the expression-inhibitory cells, compared to the control medium.

To determine whether such a decrease in the migration was caused due to the proliferation of the cells, an MTT assay was performed using the same medium. As a result, it was revealed that a change in the proliferation of the cells in each medium was not observed (FIG. 6B).

Therefore, it was confirmed that the expression of actinin-4 played an important role as a cancer marker protein in increasing EMT and migration of the cells through a mechanism of increasing AKT-Snail-MMP-9 or inhibiting AKT-Snail-E-cadherin

### <Example 5> Examination of Expression of β-Catenin in Actinin-4-Overexpressing Cells

To examine the expression of β-catenin in the actinin-4-overexpressing cells MDCK, the actinin-4 overexpression stabilizing cells were constructed in the MDCK cells. For this purpose, the cells were transfected with a Flag-ACTN4 plasmid, and then selected by treatment with G-418 (400 μg/mL; Sigma) for 2 weeks. After RIPA lysis was performed to extract proteins from the corresponding cells, 30 μg of the proteins were Western-blotted to confirm the expression of β-catenin (BD Biosciences) and vimentin (Santa Cruz) as a target gene of β-catenin. Here, each of the antibodies was used at a ratio of 1:3,000.

Surprisingly, it was revealed that the expression of β-catenin in the actinin-4-overexpressing cells MDCK increased, and the expression of vimentin as the target protein of β-catenin also increased (FIG. 7A).

The role of β-catenin in cells is to induce the proliferation of the cells, and usually, β-catenin is bound to E-cadherin which is involved in cell-cell binding. In this case, a decrease in E-cadherin results in a decrease in β-catenin. However, although E-cadherin disappeared since the expression of E-cadherin was inhibited in the actinin-4-overexpressing cells, the expression of β-catenin increased. Therefore, since the increase in actinin-4 was considered to be involved in stabilization of β-catenin, it was checked, whether the degradation of β-catenin by actinin-4 was inhibited when the expression of E-cadherin was reduced using siRNA.

For this purpose, si-E-cadherin (an oligonucleotide: CAGACAAAGACCAGGACUA; Bioneer) was constructed. When SiHa cells were transfected with si-E-cadherin (100 nM) and actinin-4 (1 μg) genes to inhibit the expression of E-cadherin, the expression levels of β-catenin and vimentin were determined using Western blotting. Each of the corresponding antibodies was used at a ratio of 1:3,000.

As shown in FIG. 7B, it was revealed that a decrease in β-catenin due to a decrease in E-cadherin was inhibited due to the overexpression of actinin-4.

Also, to determine whether the degradation of β-catenin by actinin-4 was inhibited, the cells were treated with cycloheximide (CHX, 40 μg/mL) as a protein translation inhibitor at different times (0 to 36 hours) to determine a change in β-catenin due to the overexpression of actinin-4. β-catenin and c-myc as a target protein of β-catenin were observed using Western blotting. Each of the antibodies was used at a ratio of 1:3,000.

As a result, it was revealed that the degradation of β-catenin was further delayed when actinin-4 was overexpressed, compared to the control (mock), and a decrease in c-myc as the target protein of β-catenin was also delayed (FIG. 7C).

The degradation of β-catenin in the cells is caused by proteosomes. Therefore, to determine whether actinin-4 inhibited the degradation of β-catenin due to the degradation of the proteosomes to induce the stabilization of β-catenin, the cells were treated with a proteosome inhibitor ALLN (20 μM/mL, Calbiocam) for 6 hours to determine whether the degradation of β-catenin by actinin-4 was inhibited using Western blotting.

Also, after actinin-4 was expressed at different concentration (0, 0.05, 0.1, 0.25 and 0.5 μg) in the HEK-293T cells, the promoter luciferase activities of cyclin D1 (0.5 CCND1) were measured.

As a result, it was revealed that, when actinin-4 was overexpressed, the proteosomal degradation of β-catenin was inhibited and β-catenin was further stabilized with an increased amount of β-catenin (FIG. 8A). Also, it was revealed that the transcriptional activities of cyclin D1 (CCND1) as the target protein of β-catenin also increased according to the concentration of the expressed actinin-4 (FIG. 8B).

### <Example 6> Examination of Proliferation of Actinin-4 Expression-Inhibitory Cells and Actinin-4-Overexpressing Cells

Since an increase in β-catenin and cyclin D1 as the target protein of β-catenin induces the proliferation of cells, the expression of actinin-4 is considered to be involved in the cell proliferation. Accordingly, the proliferation of the actinin-4 expression inhibition stabilizing cells (HeLa-KD and SiHa-KD) in the HeLa and SiHa cells was determined using a colony formation assay. The cells were seeded in a 12-well plate at 3×10³ cells/well, and kept for 5 days, and the number of the colonies was then counted. Also, a cell proliferation level was determined by a colony formation experiment using the actinin-4 overexpression stabilizing cells ME-180-OV#3. For this purpose, the cells were seeded in a 12-well plate at 3×10³ cells/well, and kept for 5 days, and the number of the colonies was then counted. The colonies were stained with 0.05% crystal violet for 24 hours, and washed with distilled water (DW). Then, images of the colonies were taken using a Nikon COOLPIX P300 digital camera (12.2 Mega-pixel; Nikon Corp., Tokyo, Japan).

As a result, it was revealed that the colony formation was suppressed in the actinin-4 expression-inhibitory cells HeLa and SiHa (FIGS. 9A and B), but the colony formation increased in the ME-180 cells in which actinin-4 was overexpressed (FIG. 9C). Therefore, the overexpression of actinin-4 was considered to cause an increase in proliferation of the cells to induce cancer formation.

As described above, since the colony formation assay showed that the cell proliferation was reduced in the actinin-4 expression-inhibitory cells, an MTT assay was performed to determine whether actinin-4 was involved in the cell proliferation in the cervical cancer cells HeLa and the SiHa-actinin-4 expression inhibition stabilizing cells. For this purpose, the actinin-4 expression-inhibitory cells HeLa and SiHa were seeded in a 96-well plate at 1×10⁴ cells/well, and the number of the cells was then counted at different times (12 to 36 hours), and plotted in a graph. In this case, the experiment was performed in triplicate. After each well was treated with 100 μl of an MTT solution (5 mg/mL, Sigma) for 6 hours, 100 mL of dimethyl sulfoxide (DMSO) was added to stop the reaction. The measurement was performed at a wavelength of 590 nm using an ELISA reader (Bio-Rad Laboratories, Inc.).

As shown in FIG. 10, it was revealed that the cell growth was reduced in both of the HeLa and SiHa cells, compared to the control.

Also, there is a propidium iodide (PI) staining assay as an experiment by which the growth or proliferation of cells can be confirmed. The PI staining assay is a method of identifying cells of G0, G1, S, and G2/M phases and counting the number of the cells as an experiment capable of checking a cell differentiation process. Assuming that the growth and proliferation of the cells increased as can be seen from the results, this experiment uses a principle that the number of the S-phase cells increases when cell division is induced whereas the number of the S-phase cells decreases when the cell proliferation is reduced. For this purpose, the HeLa and SiHa cells (the control) and the actinin-4 expression-inhibitory cells (KD) were seeded in a 100 mm dish at 2×10⁶ cells/dish, and the cells were fixed with 70% ethanol at −20° C. for an hour. Propidium iodide (Sigma) including RNase was added at a concentration of 50 μg/mL, and the fixed cells were then incubated at 37° C. for 30 minutes. After 30 minutes, the cell number (%) of the corresponding cells by each of the cell cycles was counted using FACSCAN® (BD Biosciences, FACS® Calibur).

As shown in FIG. 11, it was revealed that the percentage of the S-phase cells was reduced in the actinin-4 expression-inhibitory cells HeLa and SiHa.

From the results, it was seen that actinin-4 functioned to promote the growth of cells transformed into cancer.

### <Example 7> Examination of Formation of Tumor in Mice into which Actinin-4 Expression-Inhibitory Cells are Injected

Since the overexpression of actinin-4 increased the growth of the cells in Example 6, this effect was tested using a mouse model.

For this purpose, SiHa-control or SiHa-KD cells (3×10⁶ cells in 0.1 mL of PBS) were subcutaneously injected into laboratory nude mice in order to determine whether cancer tissues were formed in the laboratory nude mice using the actinin-4 expression-inhibitory cells and the control cells. Four-week-old male BALB/c nude mice (Orient Bio Inc.), and five mice were used for each of the cells (n=5). After cancer cells were injected into the mice, the mice were kept at a temperature of 22±2° C. and a humidity of 50±10% for 12 hours in the light and 12 hours in the dark. The size of the formed tumor was measured daily over 1 to 28 days after injection using a digital caliper, and the volume of the tumor was calculated by V=0.5×(width²×length).

Also, images of the tumors in the nude mice of the SiHa-control and the SiHa-expression-inhibitory group were taken 28 days after the cancer cell injection to compare the sizes of the tumors. The images were taken using a Nikon COOLPIX P300 digital camera (12.2 Mega-pixel; Nikon Corp., Tokyo, Japan).

Tumor tissues were extracted from the each of the groups in which the tumor was formed as described above, and images of the tumor tissues were taken to compare the sizes of the tumor tissues. The images were taken using a Nikon COOLPIX P300 digital camera (12.2 Mega-pixel; Nikon Corp., Tokyo, Japan).

Also, the sizes (using a digital caliper; V=0.5×(width²×length)) and weight (using ADVENTURER™, OHAUS Corp. USA) of the formed tumors were measured at different times to compare the body weights of the mice in each of the mouse groups after the cancer cell injection.

The SiHa cells in which the expression of actinin-4 was inhibited were injected into the mice to confirm whether the tumor was formed at different times. As a result, it was revealed that the tumor formation was inhibited in the actinin-4 expression-inhibitory cells, compared to the control cell (FIG. 12A). Also, it was confirmed whether the tumor was formed 28 days after the injection. As a result, it was revealed that the size of the tumor was also smaller than that of the control (FIG. 12B).

Also, the sizes of the tumors removed from the mice into which the actinin-4 expression-inhibitory cells were injected were compared. As a result, it was revealed that the sizes of the tumors were smaller than that of the control (FIG. 13A). Also, the tumors extracted from the mice were compared. As a result, it was revealed that the sizes and weights of the tumors were also significantly reduced, compared to those of the control (FIG. 13B). However, there was no change in body weights of the mice into which the cancer cells were injected (FIG. 13C).

The present invention can be used in a kit for diagnosing cervical cancer, or used to prevent or treat a prophylactic or therapeutic agent for cervical cancer.

What is claimed is:

1. A method of screening a drug for effectiveness in treating cervical cancer, comprising:
contacting an actinin-4 protein with a candidate compound; and
determining whether the candidate compound inhibits functions or activities of the actinin-4 protein.

2. The method of claim 1, wherein the cervical cancer is human papillomavirus type 16 or 18 positive cervical cancer.

3. The method of claim 1, wherein the actinin-4 protein is human actinin-4 protein.
